(19) United States
(12) Patent Application Publication
(10) Pub. No.: US 2023/0233224 A1
(43) Pub. Date: Jul. 27, 2023

(54) VIBRATION TRANSMISSION MEMBER AND TREATMENT TOOL

(71) Applicant: Olympus Corporation, Tokyo (JP)

(72) Inventors: Hiroshi ASHIBA, Tokorozawa-shi (JP); Yukihiko Shimamura, Yoshikawa-shi (JP)

(21) Appl. No.: 18/295,441

(22) Filed: Apr. 4, 2023

Related U.S. Application Data
(63) Continuation of application No. PCT/JP2020/038159, filed on Oct. 8, 2020.

(51) Int. Cl. A61B 17/32

(52) U.S. Cl. CPC A61B 17/320068; A61B 2017/320073; A61B 2017/320082

(57) ABSTRACT

A vibration transmission member includes: a main body that extends from a front end toward a proximal end of the vibration transmission member to define a longitudinal axis direction and that has a proximal end to which an ultrasonic transducer configured to generate ultrasonic vibration is connected; and an end effector that is installed at a front end of the main body and that has a curved shape which curves in a first direction toward the front end of the vibration transmission member, the first direction being orthogonal to the longitudinal axis direction, the end effector being configured to apply the ultrasonic vibration to a body tissue to treat the body tissue.

### CROSS-REFERENCE TO RELATED APPLICATION

This application is a continuation of International Application No. PCT/JP2020/038159, filed on Oct. 8, 2020, the entire contents of which are incorporated herein by reference.

### BACKGROUND

### 1. Technical Field

The present disclosure relates to a vibration transmission member and a treatment tool.

### 2. Related Art

In the related art, a treatment tool is known that applies energy to such a region in a body tissue which is to be treated (hereinafter, referred to as a target region), and thus treats the target region (for example, refer to Japanese Unexamined Patent Publication No. 2006-513737).

The treatment tool disclosed in Japanese Unexamined Patent Publication No. 2006-513737 is an ultrasonic treatment tool that includes a vibration transmission member transmitting ultrasonic vibrations, which are generated by an ultrasonic transducer, from a proximal end to a front end of the vibration transmission member. The vibration transmission member includes a main body and an end effector as explained below.

The main body has an elongated shape and has a proximal end to which the ultrasonic transducer is connected.

The end effector is installed at a front end of the main body, and applies ultrasonic vibrations to the target region. The end effector has a curved shape that curves in a first direction which is orthogonal to a longitudinal axis direction of the main body. More particularly, the end effector includes: a constricted portion that goes on increasing in size in a second direction toward the front end of the vibration transmission member, the second direction being orthogonal to the longitudinal axis direction and the first direction; and a wide portion that is provided on a front end side of the constricted portion and that has a size in the second direction that is greater than a size in the first direction.

### SUMMARY

In some embodiments, a vibration transmission member includes: a main body that extends from a front end toward a proximal end of the vibration transmission member to define a longitudinal axis direction and that has a proximal end to which an ultrasonic transducer configured to generate ultrasonic vibration is connected; and an end effector that is installed at a front end of the main body and that has a curved shape which curves in a first direction toward the front end of the vibration transmission member, the first direction being orthogonal to the longitudinal axis direction, the end effector being configured to apply the ultrasonic vibration to a body tissue to treat the body tissue, the end effector including a constricted portion configured to increase in size in a second direction toward a front end of the constricted portion, the second direction being orthogonal to the longitudinal axis direction and the first direction, and a wide portion that is provided on a front end side of the constricted portion and that has a size in the second direction that is greater than a size in the first direction, a stress concentration portion at which stress attributed to the ultrasonic vibration is concentrated being set on a proximal end side of the constricted portion, and a cross-sectional area adjustment portion that includes the constricted portion and that has a size along the longitudinal axis direction to be within a predetermined range being set to have a cross-sectional area orthogonal to the longitudinal axis direction to be within a predetermined range.

In some embodiments, a treatment tool includes: a cylindrical sheath; a vibration transmission member that is inserted into the sheath and that has a front end protruding out from the sheath; and an ultrasonic transducer configured to generate ultrasonic vibration, the vibration transmission member including: a main body that extends from a front end toward a proximal end of the vibration transmission member to define a longitudinal axis direction and that has a proximal end to which the ultrasonic transducer is connected; and an end effector that is installed at a front end of the main body and that has a curved shape which curves in a first direction toward the front end of the vibration transmission member, the first direction being orthogonal to the longitudinal axis direction, the end effector being configured to apply the ultrasonic vibration to a body tissue to treat the body tissue, the end effector including a constricted portion configured to increase in size in a second direction toward a front end of the constricted portion, the second direction being orthogonal to the longitudinal axis direction and the first direction, and a wide portion that is provided on a front end side of the constricted portion and that has a size in the second direction that is greater than a size in the first direction, a stress concentration portion at which stress attributed to the ultrasonic vibration is concentrated being set on a proximal end side of the constricted portion, and a cross-sectional area adjustment portion that includes the constricted portion and that has a size along the longitudinal axis direction to be within a predetermined range being set to have a cross-sectional area orthogonal to the longitudinal axis direction to be within a predetermined range.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIGS. 2 and 3 are diagrams illustrating a front end portion of a vibration transmission member;

FIG. 4 is a diagram illustrating a variation of a cross-sectional area in the front end portion of the vibration transmission member; and

FIGS. 5 and 6 are diagrams illustrating the front end portion of a vibration transmission member representing a comparative example of the vibration transmission member according to the embodiment.

### DETAILED DESCRIPTION

An illustrative embodiment (hereinafter, called an embodiment) of the disclosure is described below with reference to the accompanying drawings. However, the disclosure is not limited by the embodiment described below. Moreover, in the drawings, identical constituent elements are referred to by the same reference numerals.

Overall Configuration of Treatment System

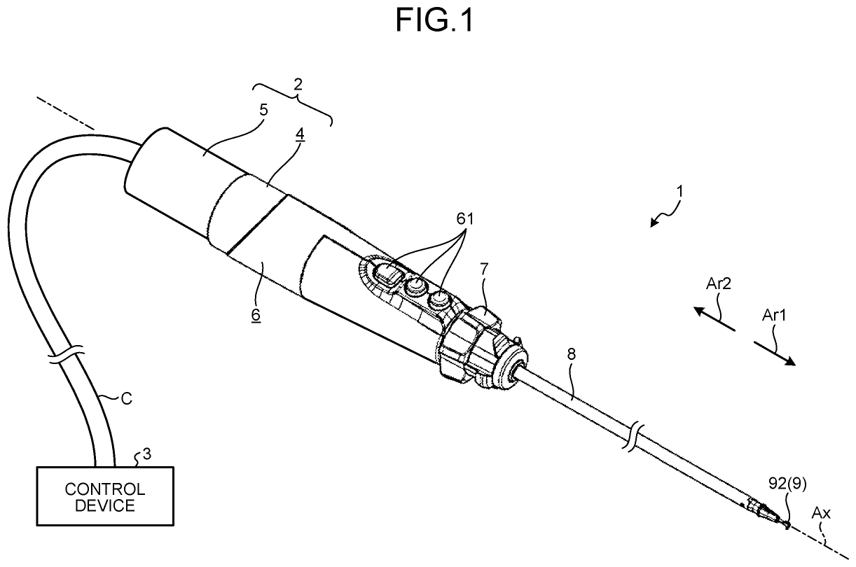

FIG. 1 is a diagram illustrating a treatment system 1 according to the embodiment.

The treatment system 1 applies treatment energy to such a region in a body tissue to be treated (hereinafter, referred to as a target region) and thus treats the target region. In the present embodiment, ultrasonic energy and high-frequency energy is used as the treatment energy. Meanwhile, examples of the treatment include coagulation of the target region and incision of the target region. As illustrated in FIG. 1, the treatment system 1 includes a treatment tool 2 and a control device 3.

Configuration of Treatment Tool

The treatment tool 2 is an ultrasonic treatment tool that applies at least the ultrasonic energy to the target region and treats the target region. As illustrated in FIG. 1, the treatment tool 2 includes a treatment tool main body 4 and an ultrasonic transducer 5.

The treatment tool main body 4 applies the treatment energy to the target region. As illustrated in FIG. 1, the treatment tool main body 4 includes a housing 6, a rotation knob 7, a sheath 8, and a vibration transmission member 9.

In the following explanation, one side along a central axis Ax of the sheath 8 is referred to as a front end side Ar1 and the other side along the central axis Ax of the sheath 8 is referred to as a proximal end side Ar2. The central axis Ax is equivalent to a longitudinal axis.

The housing 6 has a substantially cylindrical shape in a coaxial manner to the central axis Ax. The housing 6 supports the entire treatment tool main body 4.

As illustrated in FIG. 1, the housing 6 includes switches 61 that are exposed to an outside of the housing 6 and that receive treatment start operations of a user. The switches 61 output operation signals corresponding to the treatment start operations to the control device 3 via an electric cable C (see FIG. 1), which electrically connects the treatment tool 2 and the control device 3.

As illustrated in FIG. 1, the rotation knob 7 has a substantially cylindrical shape in a coaxial manner with the central axis Ax, and is installed at the front end side Ar1 of the housing 6. The rotation knob 7 receives a rotation operation performed by the user. The rotation operation is meant for rotating the vibration transmission member 9 around the central axis Ax. When the rotation operation is performed, the rotation knob 7, the sheath 8, and the vibration transmission member 9 rotate around the central axis Ax.

The sheath 8 is a cylindrical pipe made of a metallic material, and the end portion thereof at the proximal end side Ar2 is supported by the housing 6. The vibration transmission member 9 is inserted into the sheath 8.

The vibration transmission member 9 is made of an electroconductive material, and has an elongated shape extending along the central axis Ax. Moreover, the vibration transmission member 9 is inserted into the sheath 8 such that an end portion of the vibration transmission member 9 at the front end side Ar1 (i.e., an end effector 92 (explained later)) protrudes out from the sheath 8. Furthermore, a proximal end of the vibration transmission member 9 is connected to a bolted Langevin-type transducer (BLT) that constitutes the ultrasonic transducer 5. Thus, the vibration transmission member 9 transmits the ultrasonic vibrations, which are generated by the BLT, from an end portion at the proximal end side Ar2 to an end portion at the front end side Ar1. In the present embodiment, the ultrasonic vibrations represent longitudinal vibrations in a direction running along the central axis Ax.

Meanwhile, regarding the detailed shape of the front end portion of the vibration transmission member 9, the explanation is given later in a section called “shape of front end portion of vibration transmission member”.

The ultrasonic transducer 5 is inserted into the housing 6 from the proximal end side Ar2 of the housing 6, and is detachably connected to the housing 6. Although the specific illustration is not given in the drawings, the ultrasonic transducer 5 includes a BLT that generates ultrasonic vibrations according to the supply of driving signals representing the alternating-current power.

### Configuration of Control Device

The treatment tool 2 is detachably connected to the control device 3 via the electric cable C. According to an operation signal (a treatment start operation) input from the switches 61 via the electric cable C, the control device 3 comprehensively controls operations of the treatment tool 2 as explained below.

The control device 3 outputs a driving signal to the BLT, which constitutes the ultrasonic transducer 5, via the electric cable C. This causes the BLT to generate ultrasonic vibrations (longitudinal vibrations). The vibration transmission member 9 vibrates at a desired amplitude of vibration due to the longitudinal vibrations. As a result, from the end portion at the front end side Ar1 of the vibration transmission member 9, ultrasonic vibrations get applied to the target region that is in contact with the end portion at the front end side Ar1 of the vibration transmission member 9. In other words, ultrasonic energy gets applied to the target region from the end portion of the vibration transmission member 9 on the front end side Ar1.

Meanwhile, the control device 3 is connected to a return electrode (not illustrated) via an electric cable (not illustrated). The return electrode is attached to an outer surface of a subject. The control device 3 outputs a high-frequency signal representing a high-frequency power in between the vibration transmission member 9 and the return electrode via the abovementioned electric cable and via the electric cable C. This causes a high-frequency current to flows in the target region that is positioned in between the end portion at the front end side Ar1 of the vibration transmission member 9 and the return electrode. In other words, high-frequency energy gets applied to the target region from the end portion of the vibration transmission member 9 on the front end side Ar1.

Shape of front end portion of vibration transmission member

Given below is the explanation about the shape of the front end portion of the vibration transmission member 9.

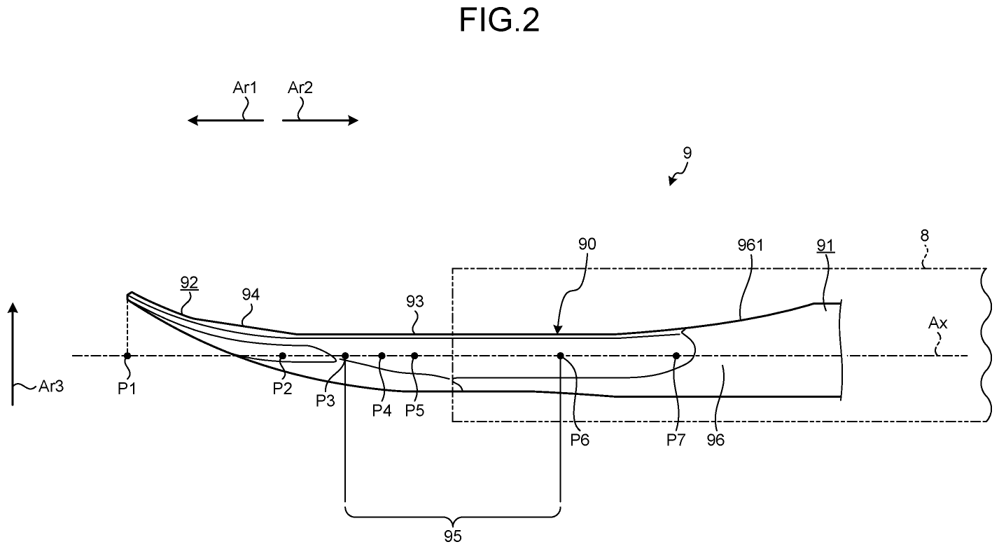

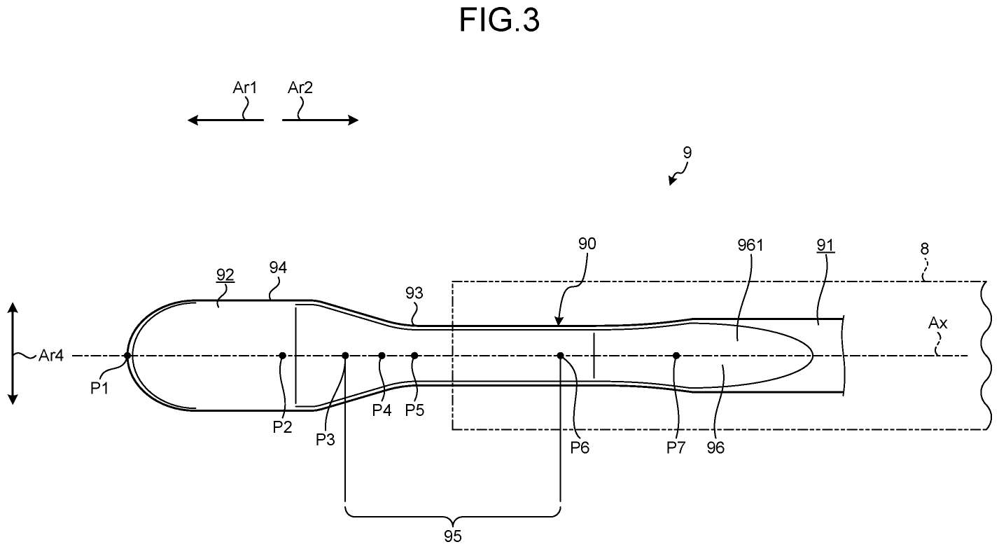

FIGS. 2 and 3 are diagrams illustrating the front end portion of the vibration transmission member 9. More particularly, FIGS. 2 and 3 are diagrams illustrating the front end portion of the vibration transmission member 9 from two directions that are orthogonal to the central axis Ax. With reference to FIG. 2, an upward direction orthogonal to the central axis Ax is referred to as a first direction Ar3. With reference to FIG. 3, a vertical direction orthogonal to the central axis Ax is referred to as a second direction Ar4.

As illustrated in FIGS. 2 and 3, the vibration transmission member 9 includes a main body 91 and the end effector 92.

The main body 91 has an elongated shape extending along the central axis Ax. The ultrasonic transducer 5 is connected to a proximal end of the main body 91.

The end effector 92 is installed at a front end of the main body 91 and applies ultrasonic vibrations to the target region. As illustrated in FIG. 2, the end effector 92 has a curved shape that curves in the first direction Ar3 toward the front end side Ar1. Moreover, the end effector 92 decreases in size in the first direction Ar3 toward the front end side Ar1. The end effector 92 includes a constricted portion 93 and a wide portion 94.

As illustrated in FIG. 3, the constricted portion 93 increases in size in the second direction Ar4 toward the front end of the vibration transmission member 9.

The wide portion 94 is provided on the front end side Ar1 of the constricted portion 93, and has a flattened shape with a greater size in the second direction Ar3 than a size in the first direction Ar3.

As explained above, the end effector 92 is configured to have a shape of a spatula.

Variation in cross-sectional area in front end portion of vibration transmission member

Given below is the explanation about the variation of the cross-sectional area in the front end portion of the vibration transmission member 9.

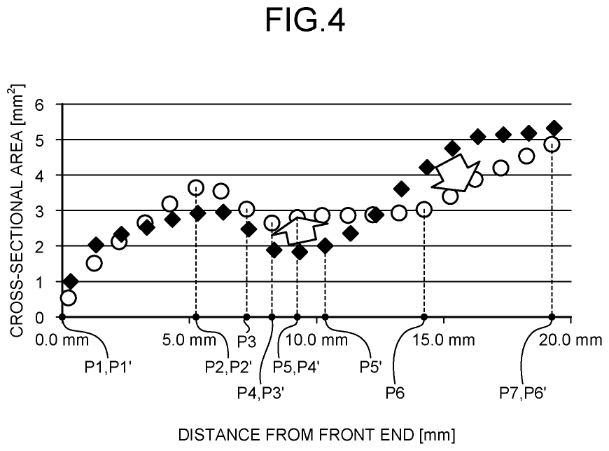

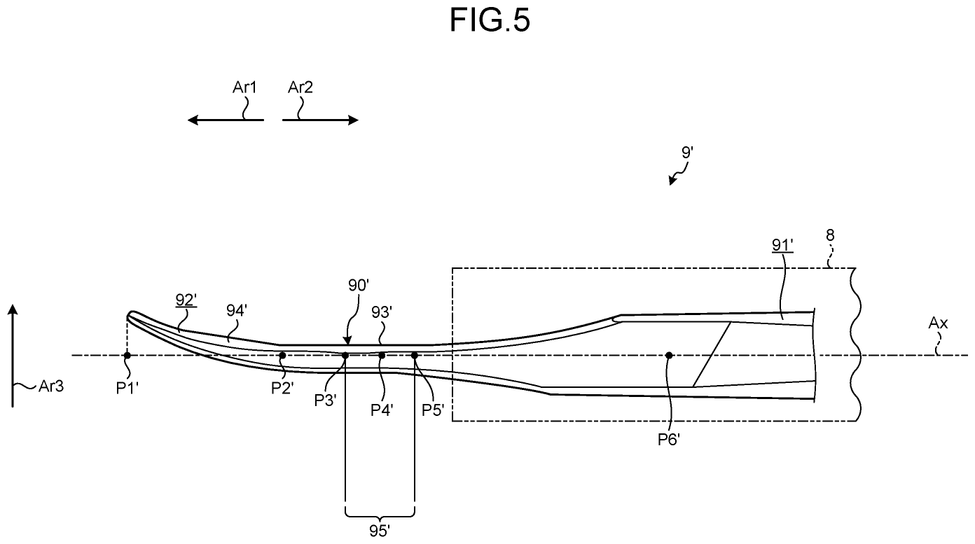

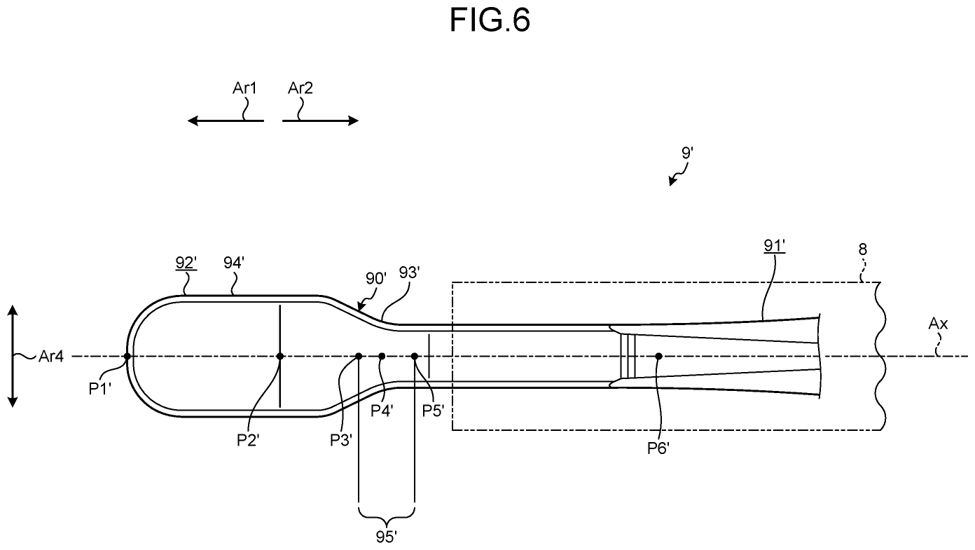

FIG. 4 is a diagram illustrating the variation of the cross-sectional area in the front end portion of the vibration transmission member 9. More particularly, FIG. 4 is a graph in which a vertical axis represents the cross-sectional area orthogonal to the central axis Ax and a horizontal axis represents a distance from the front end of the vibration transmission member 9. Moreover, in FIG. 4, the variation of the cross-sectional area in the front end portion of the vibration transmission member 9 is illustrated using white circles. Furthermore, in FIG. 4, a variation of a cross-sectional area in a front end portion of a vibration transmission member 9′, which represents a comparative example of the vibration transmission member 9, is illustrated using black quadrangular shapes. FIGS. 5 and 6 are diagrams illustrating the front end portion of the vibration transmission member 9′ representing the comparative example.

In the vibration transmission member 9′, regarding the identical configuration to the vibration transmission member 9, the same reference numerals are used along with the single quote mark. Meanwhile, reference numerals P1 to P7 illustrated in FIGS. 2 and 3 correspond to distances P1 to P7 from the front end of the vibration transmission member 9 as illustrated in FIG. 4. In an identical manner, reference numerals P1′ to P6′ illustrated in FIGS. 5 and 6 correspond to distances P1′ to P6′ from the front end of the vibration transmission member 9′ as illustrated in FIG. 4. In the vibration transmission member 9, from among the positions of the nodes of longitudinal vibrations, the frontmost node position that is closest to the front end side Ar1 is at the distance of 33.2 mm from the front end of the vibration transmission member 9. In comparison, in the vibration transmission member 9′ representing the comparative example, the frontmost node position is at the distance of 34.3 mm from the front end of the vibration transmission member 9′.

In the vibration transmission member 9′ representing the comparative example, as illustrated in FIG. 4, in the vicinity of a constricted portion 93′ (at the distance P4′ from the front end of the vibration transmission member 9′), there is an increase in the variation of the cross-sectional area. For that reason, in a stress concentration portion 90′ (at the distance of P3′ from the front end of the vibration transmission member 9′) positioned in the front end portion in the vicinity of the constricted portion 93′, there occurs concentration of stress attributed to lateral vibrations that is generated due to the curved shape in the end effector 92′.

As compared to the vibration transmission member 9′ representing a comparative example, in the vibration transmission member 9, as illustrated by arrows in FIG. 4, the variation of the cross-sectional area is kept at a moderate level by increasing the cross-sectional area in the vicinity of the constricted portion 93 (at the distance P5 from the front end of the vibration transmission member 9) and by reducing the cross-sectional area in the vicinity of the portion between the distances P6 from the front end of the vibration transmission member 9 and P7 from the front end of the vibration transmission member 9.

Herein, the region between the distance P3 from the front end of the vibration transmission member 9 and the distance P6 from the front end of the vibration transmission member 9 (i.e., the region having the size of about 7.0 mm in the direction along the central axis Ax) has a change rate of the cross-sectional area within +15% with reference to the cross-sectional area at the distance P5 representing the minimum cross-sectional area. That region corresponds to a cross-sectional area adjustment portion 95 (see FIGS. 2 and 3). Meanwhile, in the vibration transmission member 9′ representing a comparative example, since there is an increase in the variation of the cross-sectional area in the vicinity of the constricted portion 93′, a cross-sectional area adjustment portion 95′ that corresponds to the cross-sectional area adjustment portion 95 is small in size (about 2.0 mm) between the distance P3′ from the front end of the vibration transmission member 9′ and the distance P5′ from the front end of the vibration transmission member 9′ in the direction along the central axis Ax.

Moreover, a portion that is on the proximal end side Ar2 with respect to the distance P6 from the front end of the vibration transmission member 9 and that is connected to the cross-sectional area adjustment portion 95 decreases in cross-sectional area toward the front end side Ar1. That portion corresponds to a cross-sectional area decreasing portion 96. In the cross-sectional area decreasing portion 96, a slanted portion 961 that is positioned on the side of the first direction Ar3 and that is slanted to an opposite side of the first direction Ar3 toward the front end side Ar1 is arranged. The cross-sectional area in the cross-sectional area decreasing portion 96 decreases toward the front end side Ar1 due to the slanted portion 961.

As a result of having the shape as explained above, in the vibration transmission member 9, a stress concentration portion 90 (at the distance P6 from the front end of the vibration transmission member 9) is positioned to the adjacent side to the frontmost node position, and is set on the proximal end side Ar2 of the constricted portion 93 as illustrated in FIGS. 2 and 3. The stress concentration portion 90 gets positioned inside the sheath 8.

According to the embodiment described above, it becomes possible to achieve the following effects.

In the vibration transmission member 9 according to the embodiment, as a result of providing the cross-sectional area adjustment portion 95 and keeping the variation in the cross-sectional area at a moderate level, the stress concentration portion 90 is positioned close to the frontmost node position and is set on the proximal end side Ar2 of the constricted portion 93. For that reason, it becomes possible to alleviate the concentration of stress in the front end portion in the vicinity of the constricted portion 93. Moreover, since the stress concentration portion 90 is positioned inside the sheath 8, it becomes possible to avoid the collision of other tools, such as forceps, with the stress concentration portion 90.

Moreover, in the vibration transmission member 9, the cross-sectional area decreasing portion 96 is provided. Hence, along with the cross-sectional area adjustment portion 95, the variation occurring in the cross-sectional area is kept at a moderate level. For that reason, the stress concentration portion 90 can be further positioned to the adjacent side to the frontmost node position.

Particularly, because of the slanted portion 961, the cross-sectional area of the cross-sectional area decreasing portion 96 becomes smaller toward the front end side Ar1. Hence, it becomes possible to maintain balance with the curved shape of the end effector 92, and to alleviate the lateral vibrations generated due to the curved shape.

### OTHER EMBODIMENTS

Till now, the explanation was given about the embodiment of the disclosure. However, the disclosure is not limited by the embodiment described above.

In the embodiment described above, the treatment tool according to the disclosure is so configured that ultrasonic energy as well as high-frequency energy is applied to the target region. However, that is not the only possible case. Alternatively, the configuration can be such that only ultrasonic energy is applied.

Because of the vibration transmission member and the treatment tool according to the disclosure, it becomes possible to alleviate the concentration of stress in the front end portion in the vicinity of the constricted portion.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A vibration transmission member comprising:
a main body that extends from a front end toward a proximal end of the vibration transmission member to define a longitudinal axis direction and that has a proximal end to which an ultrasonic transducer configured to generate ultrasonic vibration is connected; and
an end effector that is installed at a front end of the main body and that has a curved shape which curves in a first direction toward the front end of the vibration transmission member, the first direction being orthogonal to the longitudinal axis direction, the end effector being configured to apply the ultrasonic vibration to a body tissue to treat the body tissue,
the end effector including
a constricted portion configured to increase in size in a second direction toward a front end of the constricted portion, the second direction being orthogonal to the longitudinal axis direction and the first direction, and
a wide portion that is provided on a front end side of the constricted portion and that has a size in the second direction that is greater than a size in the first direction,
a stress concentration portion at which stress attributed to the ultrasonic vibration is concentrated being set on a proximal end side of the constricted portion, and
a cross-sectional area adjustment portion that includes the constricted portion and that has a size along the longitudinal axis direction to be within a predetermined range being set to have a cross-sectional area orthogonal to the longitudinal axis direction to be within a predetermined range.

2. The vibration transmission member according to claim 1, wherein the end effector is configured to decrease in size in the first direction toward the front end of the vibration transmission member.

3. The vibration transmission member according to claim 1, wherein the main body includes a cross-sectional area decreasing portion configured to decrease in cross-sectional area orthogonal to the longitudinal axis direction toward the front end of the vibration transmission member, the cross-sectional area decreasing portion being connected to a proximal end of the cross-sectional area adjustment portion.

4. The vibration transmission member according to claim 3, wherein the cross-sectional area decreasing portion includes a slanted portion that is positioned on a curved side in which the end effector is curved, the slanted portion being slanted to an opposite side of the curved side toward the front end of the vibration transmission member.

5. A treatment tool comprising:
a cylindrical sheath;
a vibration transmission member that is inserted into the sheath and that has a front end protruding out from the sheath; and
an ultrasonic transducer configured to generate ultrasonic vibration,
the vibration transmission member including:
a main body that extends from a front end toward a proximal end of the vibration transmission member to define a longitudinal axis direction and that has a proximal end to which the ultrasonic transducer is connected; and
an end effector that is installed at a front end of the main body and that has a curved shape which curves in a first direction toward the front end of the vibration transmission member, the first direction being orthogonal to the longitudinal axis direction, the end effector being configured to apply the ultrasonic vibration to a body tissue to treat the body tissue,
the end effector including
a constricted portion configured to increase in size in a second direction toward a front end of the constricted portion, the second direction being orthogonal to the longitudinal axis direction and the first direction, and
a wide portion that is provided on a front end side of the constricted portion and that has a size in the second direction that is greater than a size in the first direction,
a stress concentration portion at which stress attributed to the ultrasonic vibration is concentrated being set on a proximal end side of the constricted portion, and
a cross-sectional area adjustment portion that includes the constricted portion and that has a size along the longitudinal axis direction to be within a predetermined range being set to have a cross-sectional area orthogonal to the longitudinal axis direction to be within a predetermined range.

6. The treatment tool according to claim 5, wherein
the constricted portion is positioned on an outside of the sheath, and
the stress concentration portion is positioned inside the sheath.
